(19) United States
(12) Patent Application Publication
(10) Pub. No.: US 2003/0176860 A1
(43) Pub. Date: Sep. 18, 2003

(54) Operation aid system

(75) Inventor: Kazuo Shimura, Kaisei-machi (JP)

Correspondence Address: SUGHRUE MION, PLLC, 2100 PENNSYLVANIA AVENUE, N.W., WASHINGTON, DC, 20037, US

(73) Assignee: FUJI PHOTO FILM CO., LTD.

(21) Appl. No.: 10/389,897

(22) Filed: Mar. 18, 2003

(30) Foreign Application Priority Data
Mar. 18, 2002 (JP) 074214/2002

(51) Int. Cl. A61B 17/56

(52) U.S. Cl. 606/53

(57) ABSTRACT

A template of artificial bone to be used in an operation is selected among a plurality of different templates of artificial bone and the selected template of artificial bone is input. Operating plan information is input through a keyboard and a mouse. The selected template and the operating plan information are displayed as an image.

### BACKGROUND OF THE INVENTION

1. Field of the Invention

This invention relates to an operation aid system which aids an operator in performing an operation by allowing the operator to select artificial bone to be employed in the operation and to display the selected artificial bone on a computer screen.

2. Description of the Related Art

In the orthopedic surgery, the oral surgery and the like, when a patient loses a part of a bone due to disease or accident or by a surgical removal as treatment for malignant tumor, the lost part of the bone has been replaced by his or her own bone of another part.

Recently, artificial bone (artificial hard tissue) of stainless steel, cobalt-chrome series alloy, metal such as titanium, aluminum or zirconium, oxide of these metals, ceramic such as apatite, or composite material of these materials has been put into practical use as substitution for the lost part of the bone.

Artificial bone which is to be embedded in place of the lost part of the bone is produced on the basis of a standard model which is selected, on the basis of the shape of the part to be replaced by the artificial bone, out of a plurality of standard models which have been prepared, in advance, in different shapes. To produce artificial bone on the basis of a standard model not always means that the artificial bone to be produced is a perfect copy of the standard model but sometimes means that the artificial bone to be produced is obtained by slightly modifying the standard model.

When selecting the standard model, conventionally, transparent templates modeled on contours of standard models are superposed on an image (e.g., an X-ray image) of the lost part of the bone in sequence and whether each of the templates conforms to the lost part of the bone is visually checked.

However, since the standard models are prepared at intervals of 1 mm of the diameter of the bone head (the head of the bone) or the length of the bone shaft or the bone holder and at intervals of 10 of the angle between the bone shaft and the bone holder, the number of the standard models is numerous, and accordingly, to select one template in the conventional way is a time-consuming and labor-consuming process.

Further, since being manually superposed on the image of the lost part of the bone in a limited time interval, each template cannot be accurately aligned with the image of the lost part and an optimal standard model or template cannot be constantly selected.

Further, since numerous templates must be kept at hand, management of the templates is troublesome.

### SUMMARY OF THE INVENTION

In view of the foregoing observations and description, the primary object of the present invention is to provide an operation aid system which aids an operator in performing an operation by causing the operator to perform selection of a suitable standard model and works in an operating plan by the use of a computer, thereby improving the efficiency and accuracy of the operating plan.

In accordance with the present invention, there is provided an operation aid system comprising a template selection means which selects a template of artificial bone to be used in an operation among a plurality of different templates of artificial bone and inputs the selected template of artificial bone, an input means through which operating plan information is input, and an image display means which displays the template selected by the template selection means and the operating plan information input through the input means as an image.

The “operating plan information” includes, for instance, the kind of the affected part, the kind of operation, the kind of the artificial bone, data on the shape of the artificial bone, the position in which the artificial bone is to be positioned, and the bone cutting position.

The “artificial bone” as used here need not be limited to that for the human body but may be artificial bone for an animal body.

The image display means may display, for instance, the image of the affected part of the bone and the template selected by the template selection means superposed one on another on the screen of the display means.

It is preferred that the operation aid system of the present invention further comprises a calculating means which calculates a leg length difference, a leg extension and/or a leg contraction on the basis of the operating plan information input through input means.

The “leg length difference” means the difference between the left and right legs of the patient, and the “leg extension” and the “leg contraction” mean the difference of the lengths of the leg between before and after the operation.

It is preferred that the operation aid system of the present invention further comprises a drawing means which extracts and draws the profile of the affected part of the bone, calculates the position where the affected part of the bone is expected to be shifted after the operation on the basis of information on the position of the template and displays the profile of the affected part of the bone in the expected position.

It is further preferred that the template selection means makes a message representing that no template is suitable or no template can be selected when a template which it selects is not suitable for reasons of combination.

It is further preferred that the operation aid system of the present invention further comprises a hard copy output means which outputs as a hard copy the image displayed by the image display means and/or information on the operating plan.

The “information on the operating plan” includes, for instance, information for ordering artificial bone corresponding to the template selected by the template selection means such as the name of the maker, the name of series to which the artificial bone belongs, the size of the artificial bone and accessories, position where the artificial bone is to be positioned, information for adjusting, for instance, the neck length of the bone, information for performing the operation such as the bone cutting position, images representing the profiles of the template and the affected part, and substantially exact size images of the template and the affected part.

It is preferred that the operation aid system in accordance with the present invention further comprises an order information output means which outputs, for instance, a data file on information for ordering artificial bone corresponding to the template selected by the template selection means, e.g., maker, model number, number, delivery time, place of delivery.

It is preferred that the operation aid system in accordance with the present invention further comprises a template data storage means in which template data representing a plurality of templates corresponding to a plurality of artificial bones of different shapes is stored, and the template data storage means be able to download template data from a network or media and to store the template data so that stored template data (the template data which has been stored) can be corrected on the basis of information published by artificial bone makers on the internet or through a media, or template data representing new artificial bones can added to the stored template data, which makes it unnecessary to manually input such template data and makes it feasible to rapidly and accurately update the template data.

Preferably, the template data is stored by maker information, series information, size information and the like, or such information is attached to each template data.

When template data storage means is used as a common data base installed in a network, the template data can be used through a plurality of terminals.

It is preferred that the operation aid system in accordance with the present invention further comprises a template creating means which can automatically translate the file format of electronic data to a file format which can be handled by other application software; display raster data or CAD data read by an image scanner and automatically or manually create template data on the basis of the raster data or the CAD data; or store (register) in a template data storage means the created template data in correlation with maker information, series information, size information and the like.

The template data used in the operation aid system in accordance with the present invention is preferably prepared not only for storing images of the templates as electronic data but also for facilitating retrieval and shape matching. For example, the template data preferably comprises an image file in which a plurality of pieces of image data each representing one template are recorded and a shape data file in which a plurality of pieces of shape data (e.g., vector data) each representing a feature of the shape of a template are recorded in correlation with the image data for the template.

The template data comprising the image data and the shape data may be provided recorded (stored) on a computer-readable recording medium.

In the operation aid system of the present invention, since a template of artificial bone to be used in the operation is selected among a plurality of different templates of artificial bone by the template selection means and the template and the operating plan information are displayed, planning of operation which is apt to be complicated can be smoothly performed. Further, by displaying the bone image and the image of the selected template superposed one on another, whether the selected template is really suitable for the bone of the affected part can be judged observing the superposed image and the operation can be performed observing the superposed image.

When the operation aid system of the present invention further comprises a hard copy output means which outputs as a hard copy the image displayed by the image display means and/or information on the operating plan, the image of the template of artificial bone to be used and/or the information on the operating plan to be performed can be output on paper or film. Accordingly, the paper or the film can be filed as a record, another piece of information can be manually added to the paper or the film and/or the operator can refer to the paper or the film during operation.

Further, when the operation aid system of the present invention further comprises a calculating means which calculates a leg length difference, a leg extension and/or a leg contraction on the basis of the operating plan information input through input means, selection of the template and/or adjustment of the position of the template can be effected observing these values and planning of the operation can be better performed.

Further, when the operation aid system in accordance with the present invention further comprises a template data storage means in which template data representing a plurality of templates corresponding to a plurality of artificial bones of different shapes is stored, since template data representing a plurality of templates representing a plurality of artificial bones of different shapes is stored in the form of electronic data instead of storing at hand a lot of real templates, management of the templates which has been necessary becomes unnecessary. Further, when the template data storage means is able to download template data from a network or media and to store the template data so that stored template data can be corrected on the basis of information published by artificial bone makers on the internet or through a media, or template data representing new artificial bones can added to the stored template data, which makes it unnecessary to manually input such template data and makes it feasible to rapidly and accurately update the template data.

Further, when the operation aid system of the present invention further comprises an order information output means which outputs, information for ordering artificial bone corresponding to the template selected by the template selection means, an automatic artificial bone order system which automatically orders artificial bone as soon as the template selection processing is completed can be realized. When the system is incorporated in a network such as using the internet, the user such as a doctor, a medical engineer and the like can directly trade with a maker or an agency without intervening therebetween a broker.

### BRIEF DESCRIPTION OF THE DRAWINGS

FIG. 1 is a block diagram showing an operation aid system in accordance with an embodiment of the present invention,

FIG. 2 is a schematic view showing an example of the image displayed on the screen,

FIG. 3 is a view for illustrating the measurement by the fundamental measurement means,

FIG. 4 is a view for illustrating selection and adjustment of the CUP template,

FIG. 5 is a view for illustrating movement of the CUP template,

FIG. 6 is a view illustrating measurement of the diameter of a femur medullary cavity,

FIG. 7 is a view for illustrating selection and adjustment of the STEM,

FIG. 8 is a view for illustrating drawing a profile of a femur as an affected part, and

FIG. 9 is a view for illustrating movement of the image of the profile of the femur after the operation.

### DESCRIPTION OF THE PREFERRED EMBODIMENTS

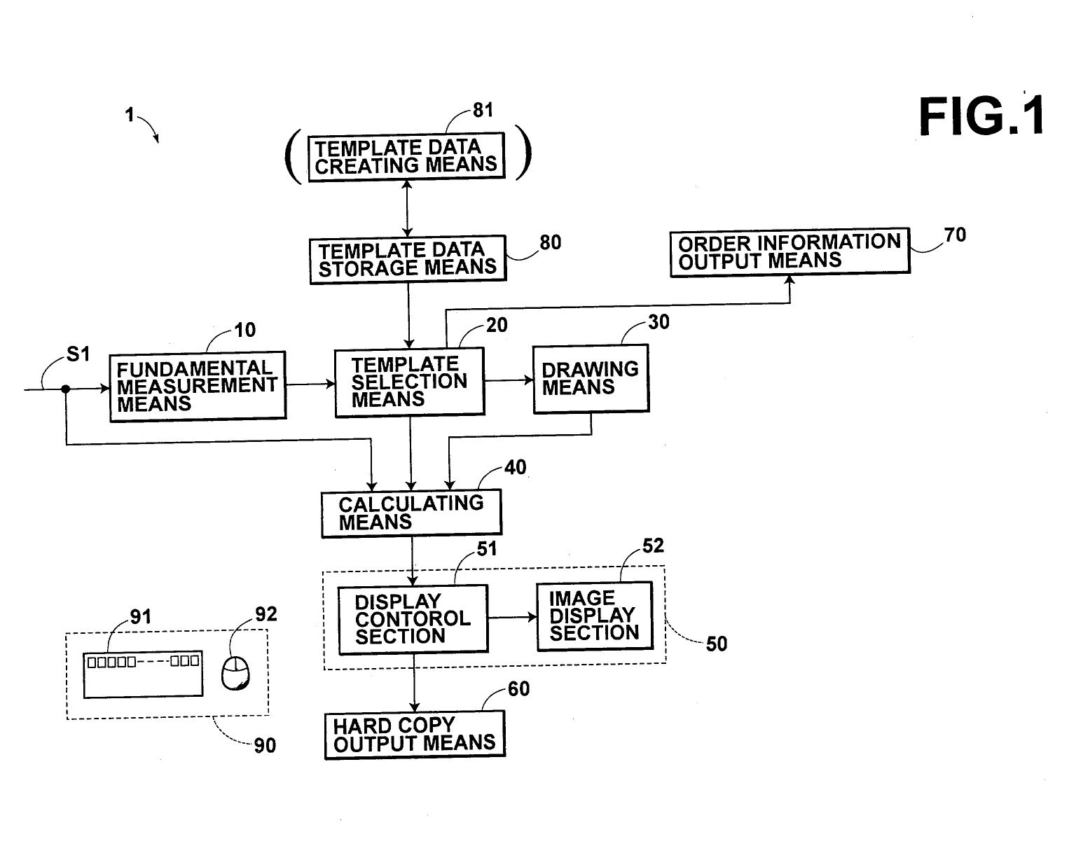

In FIG. 1, an operation aid system 1 in accordance with an embodiment of the present invention comprises a fundamental measurement means 10 which obtains fundamental data of a bone image, a template selection means 10 which selects and adjust a template of artificial bone to be used in the operation, a drawing means 30 which draws and moves a profile of the affected part to confirm the position of the affected part after the operation, a calculation means 40 which calculates various values for operation planning on the basis of information input through an input means 90 to be described later, an image display means 50 which displays the bone image, the values, an image of the template selected by the template selection means 20, the drawn profile and the like, a hard copy output means 60 which outputs as a hard copy the image displayed by the display means 50 and/or information on the operating plan, an order information output means 70 which outputs information for ordering artificial bone corresponding to the template selected and adjusted by the template selection means 20, a template data storage means 80 in which template data representing a plurality of templates corresponding to a plurality of artificial bones of different shapes is stored, a template data creating means 81 which can create template data and store the created template data in the template data storage means 80, and the input means 90 through which operating plan information is input.

The fundamental measurement means 10 calculates fundamental data and draws additional lines necessary for operation planning on the screen on the basis of the bone image and the input information.

The template selection means 20 measures the shape of the bone on the bone image, selects a template of artificial bone conforming to the part of the bone to be replaced (the affected part of the bone) and adjusts the properties (the size, the mounting angle and the like)and the layout on the screen of the selected template. In the case where the initially selected template does not allow an optimal adjustment, the template selection means 20 may reselect a template and may effect adjustment on the basis of there selected template. The template selection means 20 may be arranged either to automatically effect the measurement of the shape of the bone and the selection of the template or to cause an operator to manually effect the measurement of the shape of the bone and the selection of the template.

The calculating means 40 calculates various values for operation planning on the basis of data obtained through the fundamental measurement means 10 and the template selection means 20 and outputs them as required. When a femur is an affected part, the values include, for instance, a leg length difference, a leg extension and a leg contraction.

The image display means 50 comprises a display control section 51 and an image display section 52. The display control section 51 controls display on a screen of the image display section the bone image, the image of the template, information on the parts of the template, the drawn profile, the calculated values and the like. The display control section 51 controls the image display section 52 to constantly display the information on the parts of the template and the calculated values so that the operator can optimally select a template and optimally adjust the selected template observing the information. The image display section 52 may comprise, for instance, a CRT or a liquid crystal panel.

The hard copy output means 60 outputs as a hard copy, the information on the operating plan, the bone image and/or the image of the template displayed by the display means 50 on paper or film and may be, for instance, a printer.

The template data storage means 80 may be, for instance, a magnetic recording device such as a hard disc for temporarily storing data obtained by reading template data recorded on a computer readable portable recording medium such as a CD-ROM or MO (magneto optical disc) or by downloading data published on a network such as the internet. When the searching speed (the speed of selection) need not be high, the template data may be read out from the recording medium or the data server on the internet every time the selection of template is executed.

The template data comprises an image file in which a plurality of pieces of image data each representing one template are recorded and a shape data file in which a plurality of pieces of template shape data each representing a feature of the shape of a template are recorded in correlation with the image data for the template.

By storing template images in the form of electronic data, whether each template conforms in shape to the bone to be replaced can be checked on the basis of electronic data and the necessity of keeping numerous templates at hand and managing the templates can be eliminated.

In this particular embodiment, vector data representing a geometrical feature is employed as the template shape data. In the case of a femur, templates are divided into CUPs and STEMs. As for the CUP, data representing a center, a CUP reference line (a flat portion when the CUP is viewed sideways) and a profile of the CUP is used. As for the STEM, data representing a STEM centerline (the centerline of the stem), a STEM reference line (the junction between the stem and the neck), a neck centerline, an offset reference point (the head on the end of the neck and a profile of the STEM is used. With this arrangement, a database which not only stores template images in the form of electronic data but also facilitates retrieval and shape matching can be constructed.

The template data creating means 81 can automatically or manually create template data on the basis of raster data or CAD data or the like read by an image scanner and store in a template data storage means 80 the created template data in correlation with maker information, series information, size information and the like. The template data creating means 81 may be a part of the operation aid system of this embodiment, though not necessary.

The input means 90 is for carrying out various operations as well as inputting the operating plan information (e.g., the kind of the affected part, the kind of operation, the kind of the artificial bone, data on the shape of the artificial bone, and the position in which the artificial bone is to be positioned) and comprises a keyboard 91, a mouse 92 and the like.

Operation of the operation aid system of this embodiment will be described, hereinbelow. In this embodiment, replacement of the bone head of the hip joint will be described by way of example. In this operation, a STEM which is a substitute of a femur and a CUP which is embedded in a pelvis to form a socket for the STEM are used.

It is assumed that a radiation image including an image of the femur is obtained as the image data by the use of a CR (computed radiography) system. That is, an X-ray is projected onto an object (a patient) from an X-ray tube so that the X-ray passing through the object impinges upon an imaging plate comprising a stimulable phosphor. Then the imaging plate is loaded in a CR apparatus (an image read-out apparatus) and an image data SI representing the radiation image (including an image of the femur) recorded on the imaging plate is read out. The image data S1 is input into the operation aid system 1 of this embodiment.

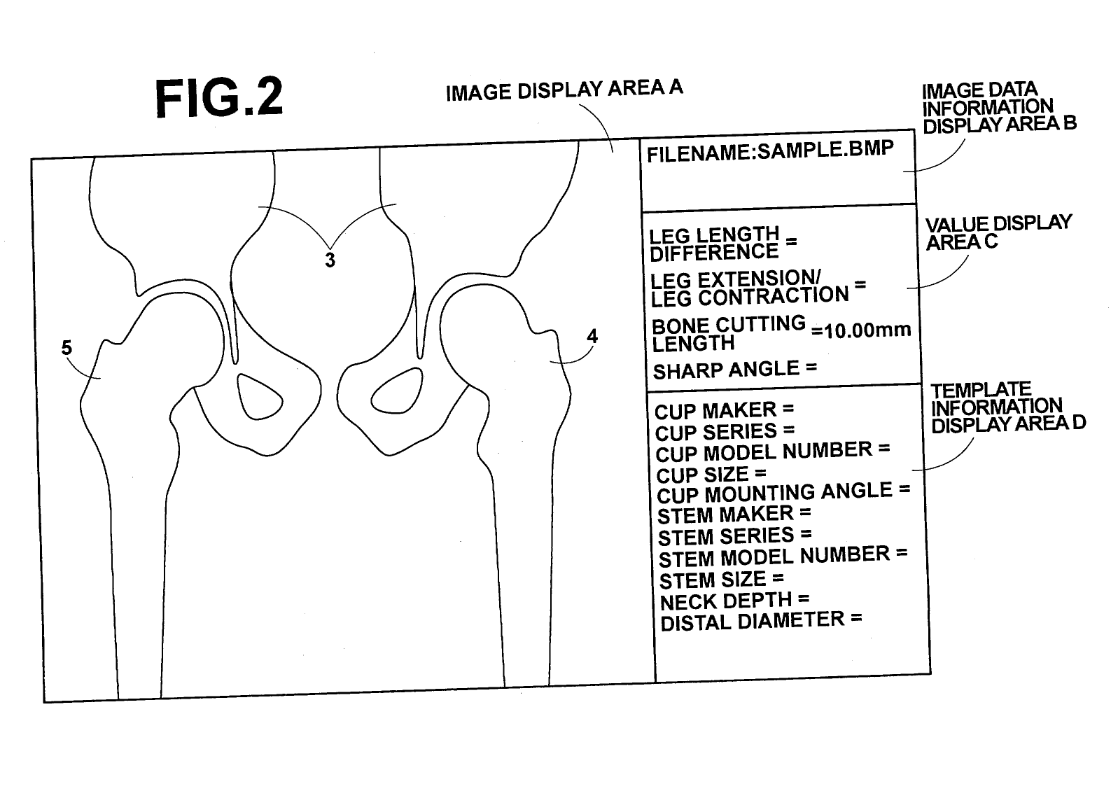

FIG. 2 shows an example of the display layout on the screen of the image display section 52 when an image including an affected femur is displayed. In this example, the screen is divided into an image display area A where the image represented by the image data SI is displayed, an image data information display area B where information on the image data SI is displayed, a value display area C where various values are displayed and a template information display area D where information on the template is displayed.

The display control section 51 causes the image display section 52 to display the image including the bone image in the image display area A on the screen thereof on the basis of the image data S1. The image in the image display area A of FIG. 2 includes a pelvis 3, a left femur 4 and a right femur 5.

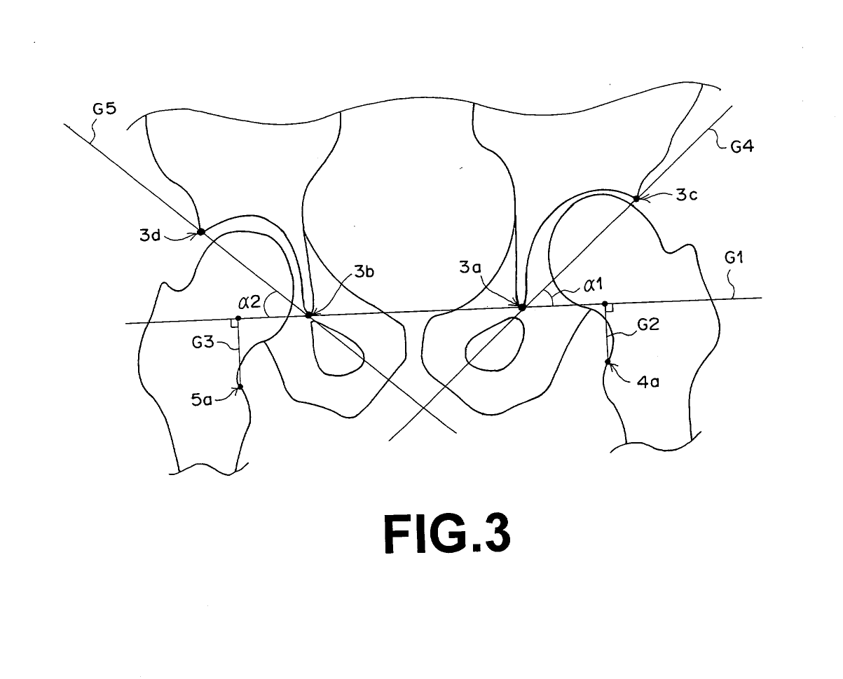

In the fundamental measurement means 10, the operator controls the mouse 92 and marks two points 3a and 3b corresponding to left and right tips (FIG. 3) of the pelvis 3. Then a straight line G1 passing through the two points is automatically drawn. Thereafter, when two points 4a and 5a corresponding to fossae of left and right lesser trochanters are marked, the perpendicular G2 from the point 4a on the line G1 and the perpendicular G3 from the point 5a on the line G1 are automatically drawn. Further when two points 3c and 3d corresponding to left and right acetabula, straight lines G4 passing through the points 3a and 3c and straight lines G5 passing through the points 3b and 3d are automatically drawn. Thereafter, the image is rotated so that the straight line G1 lies horizontal on the screen. Since the straight line passing through the left and right marks of tear drop 3 indicates the horizontal of the patient's body, the rotation of the image facilitates the visual comparison of the left and the right and measurement of distances.

The calculating means 40 calculates the leg length difference (the length of perpendicular G2—the length of perpendicular G3), and Sharp angles (angle α1 between the lines G1 and G4, angle α2 between the lines G1 and G5) and sends results of calculation to the display control section 51. The display control section 51 causes the image display section 52 to display the results of calculation in the value display area C on the screen. The leg length difference is the difference between the left and right legs of the patient. In the operation planning, the kind of the STEM and/or the position of the STEM are selected so that the lengths of the left and right legs are in a good valance after the operation. The Sharp angle is related to a tendency for the femur to be dislocated, and as the Sharp angle is an obtuse angle, the tendency for the femur to be dislocated is strengthened. In the operation planning, the kind of the CUP to be embedded in the acetabulum and the mounting angle of the CUP are determined on the basis of the Sharp angle. The left and right Sharp angles may be measured or only one of the left and right Sharp angles which undergoes operation may be measured.

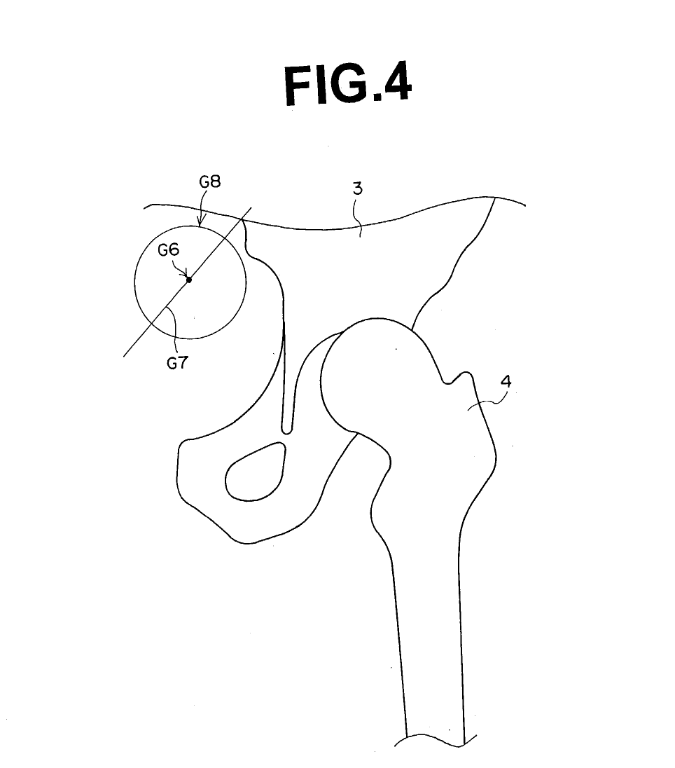

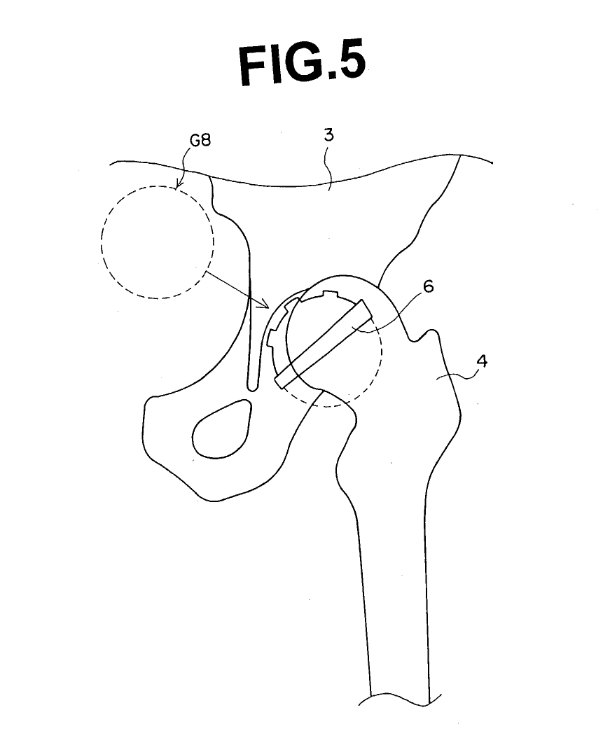

The template selection means 20 selects and adjusts the CUP and STEM. Selection and adjustment of the CUP are first done. That is, with the CUP properties displayed, a maker and a series of the CUP are selected. Then, for instance, by moving the mouse 92 with its button pressed and releasing the button, the center G6 and the radius G7 are determined (FIG. 4). Then as a circle G8 for determining the position of the CUP is displayed, the operator moves the circle G8 to a desired position on the acetabulum by drag and drop. After this operation, a CUP template 6 is displayed (FIG. 5). If necessary, the image display section 52 is caused to display again the CUP properties, and the size, the mounting angle and/or the like are readjusted. In the default, the size of CUP is equal to the diameter of the circle G8 and the mounting angle is a recommended angle (a standard angle).

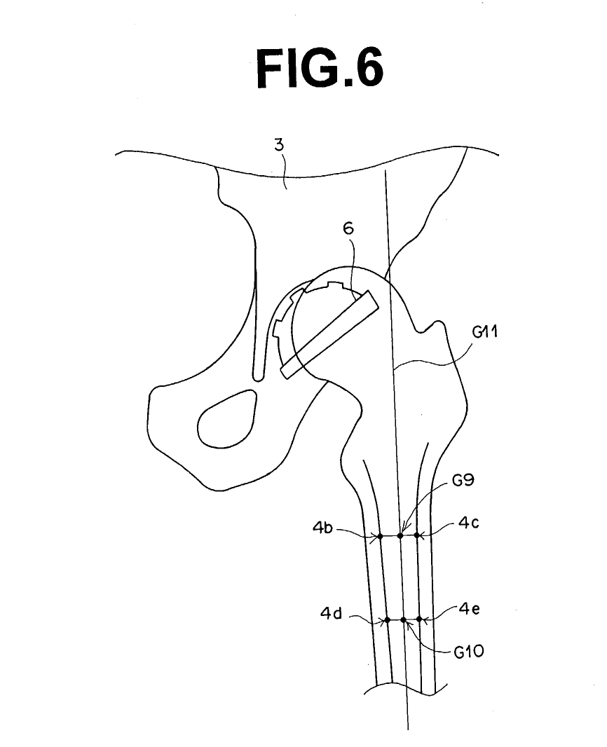

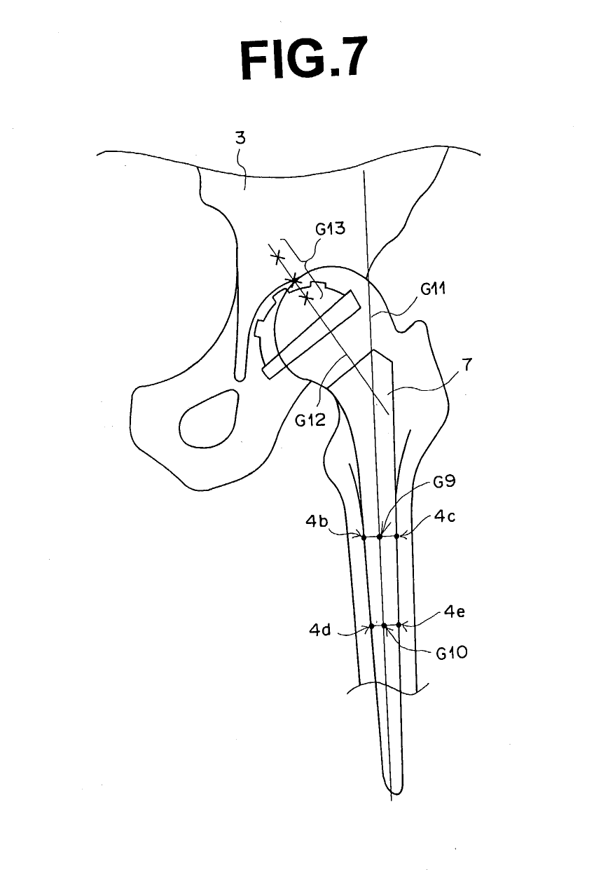

Selection and adjustment of the STEM are then done. That is, with the STEM properties displayed, a maker and a series of the STEM are selected. Thereafter two points (4b, 4c, 4d and 4e) are marked with the mouse 92 on each of the upper and lower sides of the femur and the diameter of the femur medullary cavity is measured. (FIG. 6) Thereafter, the middle points G9 and G10 between the points 4b and 4c and between the points 4d and 4e and a centerline G11 of the femur medullary cavity passing through the middle points G9 and G10 are automatically displayed. Among the templates of the STEM, the smallest one is selected and the smallest template 7 is displayed so that the centerline of the template is aligned with the centerline G11 of the femur medullary cavity. (FIG. 7) Further, a neck reference line G12 representing the axis of the neck is displayed and marks G13 representing the position of the head are displayed on the free end portion of the neck reference line G12. Since the length of the neck can be selected, one of the marks G13 may be regarded as indicating the position of the head. The template of the STEM may be moved to a desired position by drag and drop as the template of the CUP. If necessary, the image display section 52 is caused to display again the STEM properties, and the size, the depth of the neck (the length of the bone holder between the bone head and the bone stem) and/or the like are readjusted. Since the state where the selected mark G13 is aligned with the center of the CUP may be regarded as reflecting the state after the operation, the amount of leg extension or leg contraction is calculated by the calculating means 40 and displayed. Though the lesser trochanter is substantially in the same position as the bone cutting reference point (the lesser trochanter side one of the intersections of the line along which the bone is cut in order to embed the STEM and the outer edge of the STEM), the bone cutting reference point can be obtained from data on the shape of the STEM and the bone cutting length which has been determined in advance and the amount of leg extension or leg contraction may be calculated as (the amount of leg extension or leg contraction=the distance between the bone cutting reference point and the Line G1—the distance between the lesser trochanter and the Line G1). Finally, parameters including the CUP and the STEM are repeatedly adjusted watching whether the position of the CUP is proper, whether there is created a leg length difference, and the like.

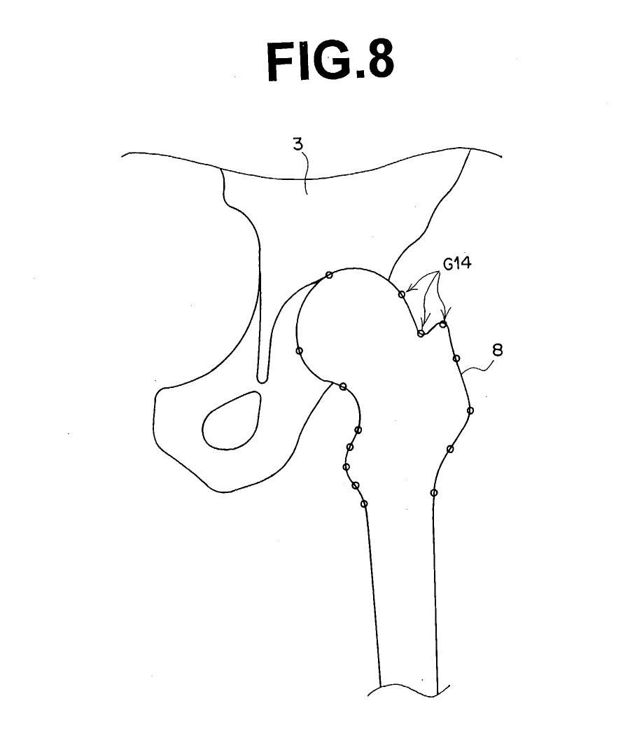

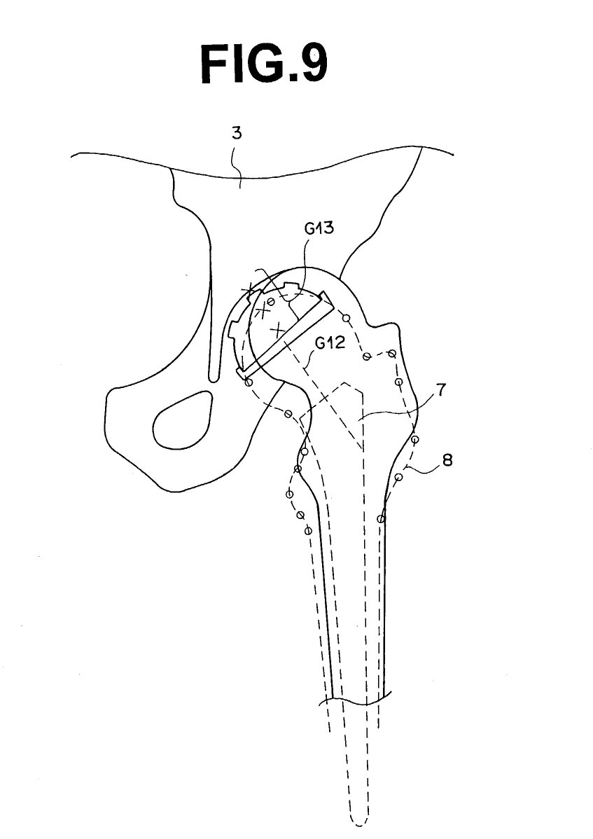

The drawing means 30 marks a plurality of points G14 on the outer edge of the femur which is the affected part to extract a profile of the femur, and draws an image 8 of a profile of the femur. (FIG. 8) The position of the femur after the operation can be obtained from information representing the position of the STEM, and the drawn profile of the femur is moved to the position of the femur after the operation as shown in FIG. 9, whereby a design drawing for operation planning can be completed.

After the template is selected and adjusted on the basis of the image data and artificial bone which can be used as a substitute of the affected part is determined in the manner described above, an order file for ordering artificial bone corresponding to the template thus selected, in which, for instance, the maker, the series, the model number (or template number), the number, the delivery time and the place of delivery are designated is generated by the order information output means 70 and sent to a maker or an agency. In the case where the operation aid system is connected to the maker or the agency by way of a network, the order file can be transferred to the maker or the agency in the form of data by way of the network.

By causing the hard copy output means 60 to output as a hard copy the image displayed by the image display means and/or information on the operating plan on paper, the paper can be filed as a record, and/or the operator can refer to the paper during operation as a reference drawing. The “information on the operating plan” includes, for instance, information necessary for ordering artificial bone corresponding to the selected template such as the name of the maker, the model number, the name of series to which the artificial bone belongs, the size of the artificial bone and accessories, position where the artificial bone is to be positioned, information for adjusting, for instance, the neck length of the bone, information for performing the operation such as the bone cutting position, images representing the template and the affected part, and substantially exact size images of the template and the affected part.

It is further preferred that the operation aid system of this embodiment be provided with a function of selectively outputting a part related to the operation so that substantially exact size images of the template and the affected part can be output on a paper of A3 size which is in a wide use. With this arrangement, a useful system can be realized without using an expensive output system which can use a paper of a larger size.

Though, in the embodiment described above, the image data S1 is obtained from a CR apparatus, the image data SI may be obtained by reading an X-ray film by an image read-out apparatus such as an image scanner. Further cross-sectional data obtained through a CT, an MRI or the like or a reconstructed three-dimensional image data can be also used.

Though the operation aid system of the embodiment described above is a type in which the operator promotes the operation planning, the operation aid system may be further provided with a navigation function so that the operation planning is guided along a preinstalled procedure or an alarm is made against an improper input. With this arrangement, even those unexperienced to operation or unfamiliar with the system can make operation planning smoothly and properly.

Further, the operation aid system of the present invention may be applied to all the operation planning without limited to the operation planning for replacement of the bone head of the hip joint. For example, it is possible to arrange the system so that the kind of the affected part or the kind of the operation can be selected, for instance, on a menu.

What is claimed is:

1. An operation aid system comprising
a template selection means which selects a template of artificial bone to be used in an operation among a plurality of different templates of artificial bone and inputs the selected template of artificial bone,
an input means through which operating plan information is input, and
an image display means which displays the template selected by the template selection means and the operating plan information input through the input means as an image.

2. An operation aid system as defined in claim 1 further comprising a hard copy output means which outputs as a hard copy the image displayed by the image display means and/or information on the operating plan.

3. An operation aid system as defined in claim 1 further comprising a calculating means which calculates a leg length difference, a leg extension and/or a leg contraction on the basis of the operating plan information input through the input means.

4. An operation aid system as defined in claim 1 further comprising a template data storage means which stores therein template data representing a plurality of templates corresponding to a plurality of artificial bones of different shapes and is able to download template data from a network or media and to store the template data.

5. An operation aid system as defined in claim 1 further comprising an order information output means which outputs information for ordering artificial bone corresponding to the template selected by the template selection means.
